(19) United States
(12) Patent Application Publication
(10) Pub. No.: US 2006/0182792 A1
(43) Pub. Date: Aug. 17, 2006

(54) Liposome-forming compositions

(76) Inventors: Holger Richardsen, Munchen (DE); Bernhard Tewes, Munchen (DE); Felix Gropp, Munchen (DE)

Correspondence Address: MICHAEL BEST & FRIEDRICH, LLP, 100 E WISCONSIN AVENUE, MILWAUKEE, WI, 53202, US

(21) Appl. No.: 10/532,576

(22) PCT Filed: Oct. 23, 2003
(86) PCT No.: PCT/EP03/11758

§ 371 Date: Jan. 4, 2006

(30) Foreign Application Priority Data
Oct. 23, 2002 (DE) 102 49 401.0

(51) Int. Cl. A61K 38/17; A61K 9/127

(52) U.S. Cl. 424/450; 514/2

(57) ABSTRACT

The invention relates to liposome-forming compositions, liposomes produced therewith and the use of said liposome-forming compositions in the production of liposomes.

This invention relates to a liposome-forming composition, the use of this composition for the formation of liposomes and liposomes which can be obtained by the use of the liposome-forming composition.

Liposomes are artificially produced uni- or multi-laminar lipid vesicles, which enclose an aqueous internal space. Generally, they are similar to biological membranes and, after attachment to the said membranes, are often therefore easily integrated into the membrane structure or they easily adhere to the cell membrane. In the first case the content of the liposome internal space is discharged into the lumen enclosed by the biological membrane. In the second case the liposomal content can be discharged into the intervening cell space. Consequently, this is a possible prerequisite for example for the paracellular transport of active substances from the intestine to the body through the intestinal wall.

Liposomes are therefore used as transport vehicles for substances, such as for example nucleic acids and pharmaceuticals. Thus, skin creams containing liposomes are produced, which transport active substances specifically into the epidermis and deeper located cell layers. For the manufacture of liposomes principally natural lecithins from soya beans or egg yolk or defined natural or artificial phospholipids, such as cardiolipin, sphingomyelin, lysolecithin and others are used.

The first liposomes were however disadvantageous in that they exhibited only a low stability. Liposomes formed from normal phospholipids forming double-layers had a very short shelf life even in the cooled state. Their storage stability could be increased through the inclusion of phosphatidic acid, but the improved stability obtained is still insufficient for many purposes. In addition, these types of conventional liposomes were not stable in acid and also not sufficiently resistant against digestion in the small intestine and were therefore neither suitable for the transport of pharmaceutical active substances which after oral administration pass through the stomach and small intestine nor for DNA transfection supported by liposomes under slightly acid pH conditions.

Therefore, attempts have been made to find new types of compounds which form liposomes. One class of substances, which has proven to be particularly promising, are tetraether lipid derivatives, such as those for example which can be obtained from natural sources. Also, there is a whole range of derivatised tetraether lipid derivatives as well as synthetic tetraether lipid derivatives. For example, reference is made here to the compounds disclosed in the German patent applications 197 36 592.2, 197 58 645.7, 100 65 561.0, 102 04 053.2 and 102 29 438.0.

These compounds generally show a satisfactory suitability for forming liposomes which overcome the aforementioned disadvantages of conventional compounds. However, it has been found that not all of the tetraether lipid derivatives discovered are suitable for forming liposomes and that furthermore the desired profiles of properties of liposomes cannot be achieved with the tetraether lipid derivatives.

The object of this invention is therefore to provide liposome-forming compositions which enable the reliable formation of liposomes, wherein at the same time an adequate mechanical and chemical stability and therefore also an extended storage capability is ensured, enabling easy and dependable usage. Furthermore, these liposome-forming compositions should exhibit suitable variability so that the required profiles of properties can be specifically and reliably set.

This object is solved according to the invention by the provision of liposome-forming compositions according to claim 1. Preferred embodiments are given in the sub-claims. Furthermore, this invention provides liposomes, which can be obtained through the use of the liposome-forming composition according to the invention. Finally, this invention makes available the use of the liposome-forming composition for the formation of liposomes. Preferred embodiments of these two developments of this invention are given in the respective sub-claims.

The compositions according to the invention are particularly suitable for forming liposomal formulations for oral administration.

The composition according to the invention contains two main constituent parts.

Firstly, the liposome-forming composition according to the invention comprises at least one lipid which is capable of forming a double layer. These lipids are known to the person skilled in the art. According to the invention, any lipids can be used which are capable of forming a double layer, wherein these lipids can be obtained from a biological source or can be produced synthetically. In this connection phospholipids of the following general formula are preferred: 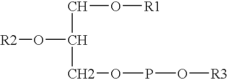
wherein R₁ and R₂ represent an aliphatic hydrocarbon chain, an acyl group or a saturated or unsaturated fatty acid radical. Preferred in this connection are alkyl groups with 10 to 20 carbon atoms, acyl groups with 10 to 20 carbon atoms and an oleoyl group, a palmitoleoyl group, an elaidoyl group, a linoleyl group, a linolenyl group, a linolenoyl group, an arachidoyl group, a vaccinyl group, a lauroyl group, a myristoyl group, a palmitoyl group or a stearoyl group. R₃ is preferably hydrogen, 2-trimenthylamino-1-ethyl, 2-amino-1-ethyl, C1-C4-alkyl, C1-C5-alkyl, substituted with a carboxy group, C2-C5-alkyl, substituted with a hydroxyl group, C2-C5-alkyl, substituted with a carboxyl group or a hydroxyl group or C2-C5-alkyl, substituted with a carboxyl group and an amino group, inositol, sphingosine or salts of said substances. Glycerides, isoprenoid liquids, steroids, sterines or sterols of lipids containing sulphur or carbohydrates can also be used as the lipid according to the invention or any other lipids forming double layers, in particular semi-protonated fluid fatty acids and similar ones. Other lipids which can be used are phosphatidylcholines, phosphatidylethanolamines, phosphatidylglycerols, phosphatidylinositols, phosphatidine acids, phosphatidylserines, sphingomyelines and other sphingophospholipids, glycosphingolipids, gangliosides and other glycolipids or synthetic lipids.

According to the invention at least one lipid of this type must be present which is capable of forming a double layer. However, it is also possible to use two or more lipids of this type in the composition according to the invention. Through appropriate selection, in particular of header groups, it is possible for example to ensure reliable attachment at the desired destination or a reliable deposition of a pharmaceutical substance to be introduced into the liposome. Lipids which form double layers and are particularly preferred are 1,2-di-stearoyl-sn-glycero-3-phosphocholin (abbreviated to DSPC in the following; the formula is given below): 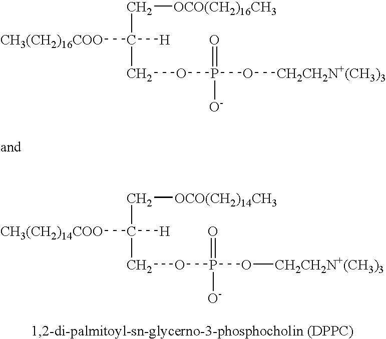
as well as their natural analogues soya phosphatidylcholin (S-PC, brand names: S100, S80 from LIPOID) or egg phosphatidytcholin (egg-PC), or the hydrogenated soya phosphatidylcholin (HSPC) and hydrogenated egg phosphatidylcholin (HEPC).

The second main component of the composition according to the invention is a tetraether lipid which extends through the double layer formed by the lipids used according to the invention.

According to the invention any tetraether lipid can be used which is suitable of substituting a pair of the double-layer forming lipids in the double layer formed by the described lipids. Preferred tetraether lipids are in particular those which are named in WO 99110337. Furthermore the tetraether lipids can be used which are disclosed in the German patent applications mentioned in the introduction.

Preferred tetraether lipids are any with the following formula (1): 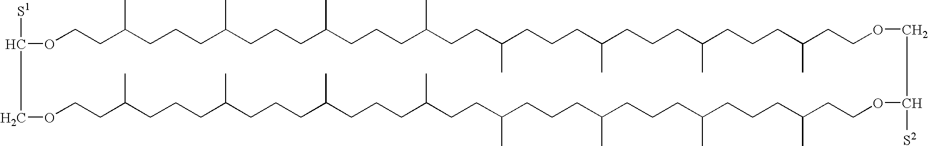
wherein S¹ and S² may be the same or different, each having the following significance: 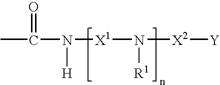
and
Y may signify —NR²R³ or —N^{⊕}R⁵R⁸;
X¹ and X² may be the same or different and are each selected independent of one another from the group which comprises a branched or unbranched alkylene or alkenylene with 2 to 20 carbon atoms;
R¹ to R⁶ may be the same or different and are each selected independently of one another from the group which comprises: hydrogen, branched or unbranched alkyl, alkenyl, aralkyl or aryl groups with 1 to 12 carbon atoms, wherein in each case one of the radicals R² to R⁶ can also comprise an antibody against cell surface molecules or a ligand for cell surface receptors; and
n may signify an integer between 0 and 10,
as well as, through the formation of pentacycles, modifications thereof formed in the tetraether basic structure.

In the preferred embodiments the substituents S¹ and S² at both ends of the tetraether lipid basic structure are the same. Starting from natural tetraether lipids, this facilitates the synthesis without the intervening use of protective groups. The identity of the substituents S¹ and S² is particularly preferred in such cases in which none of the radicals R¹ to R⁶ represents an antibody or ligand for a cell surface receptor.

In a preferred embodiment of the tetraether lipid derivative according to the invention the group X¹ represents both in S¹ and S² an alkylene or alkenylene with 2 to 10, preferably 3 to 6 carbon atoms. In particularly preferable embodiments X¹ is propylene.

The group X² is also preferably an alkylene or alkenylene with 2 to 10, preferably with 3 to 6 carbon atoms. Also for X² propylene radicals are particularly preferred.

n can signify 0 to 10. In preferred embodiments n is 0 to 3, especially preferably 0.

In further embodiments of the tetraether lipid derivative according to the invention Y signifies, both in S¹ and in S², —NR²R³. Here, R² and R³ are preferably hydrogen, branched or unbranched alkyl, alkenyl, aralkyl or aryl groups, particularly preferred are hydrogen, methyl, ethyl or propyl groups. In a further preferred embodiment Y is both in S¹ and in S² a quaternary ammonium salt, the radicals R⁴, R⁵ and R⁶ of which are also preferably hydrogen, branched or unbranched alkyl, alkenyl, aralkyl or aryl groups with 1 to 12 carbon atoms, particularly preferably hydrogen, methyl, ethyl or propyl. In each case one of the radicals R² to R⁶ can comprise an antibody against cell surface molecules or a ligand for cell surface receptors.

In particularly preferred embodiments the tetraether lipid derivative has the general formula (I) with the following stated substituents S¹ and S²:
S¹ and S²: —CO—NH—(CH₂)₃—NH₂; Compound A:
S¹ and S²; —CO—NH—(CH₂)₃—N(CH₃)₂; Compound B:
S¹ and S²: —CO—NH—(CH₂)₃—N^{⊕}(CH₃)₃. Compound C:

According to the invention the presence of at least one tetraether lipid is specified as mandatory. However, here too, mixtures of various tetraether lipids can be used. Again, through a suitable selection of the header groups, the profile of properties of the liposomes formed from the composition according to the invention can be specifically set, as already described above in conjunction with the lipids which form double layers.

A particularly preferred tetraether lipid Is a glycerol calditol tetraether lipid (abbreviated in the following to GCTE), the formula of which is shown below.

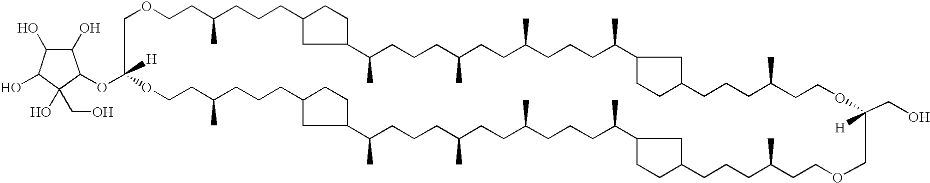

A further preferred tetraether lipid is shown below in the formula:

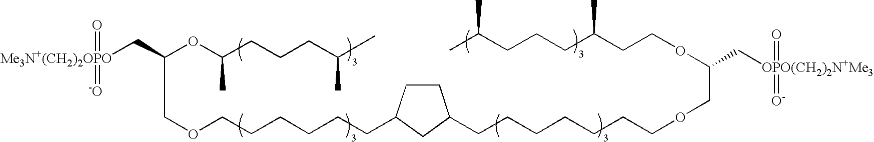

Further preferred openchained tetraether lipids are those which are mentioned in the German applications 102 04 053.2 and 102 29 438.0, which are included here in their entire extent through this reference, wherein the following compound is particularly preferred:

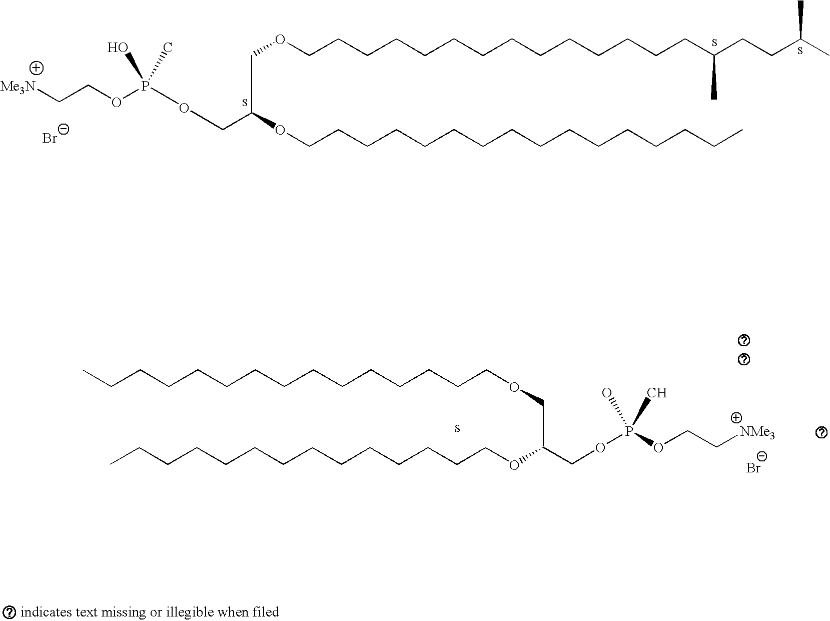

A tetraether lipid, which is used particularly preferably in combination with GCTE, is a tetraether lipid with cationic header groups, wherein the compound AF1 is preferred, the formula of which is shown below.

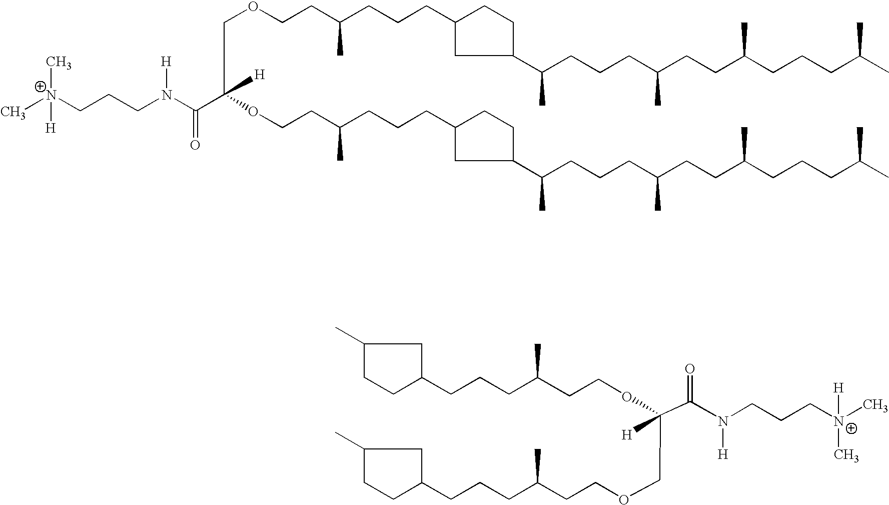

Although the composition according to the invention can contain any mixing ratios of lipids forming double layers and tetraether lipids, it has been shown that it is preferable if the molar ratio of lipids forming double layers to tetraether lipids lies in the range from 1:5 to 10:1, more preferably in the range from 2.5:1 to 5:1 and most preferably in the range from 2.5:1 to 3.5:1. If in addition a tetraether lipid, as defined above, is present with cationic header groups (as AF1), then preferably a molar ratio of double-layer forming lipid : tetraether lipid : tetraether lipid with cationic header groups of (10 to 4):(2 to 1):(1 to 0.2) set, wherein a ratio of 6:1.5:0.5 is particularly preferred. A particularly preferred combination according to the invention here is either a composition of DSPC and GCTE, with a molar ratio DSPC:GCTE of 3:1 or a composition of DSPC and GCTE and AF1 with a molar ratio DSPC:GCTE:AF1 of 6:1.5:0.5.

The liposome-forming composition according to the invention can, apart from the main constituents described above, also contain additional constituents. These are described in the following.

The composition according to the invention can also comprise at least one preservative, preferably a microbiocide. This is preferably selected from short-chained alcohols, preferably ethyl alcohol and isopropyl alcohol, chlorobutanol, benzyl alcohol, chlorobenzyl alcohol, dichlorobenzyl alcohol, hexachlorophene, phenolic compounds, such as cresol, 4-chloro-m-cresol, p-chloro-m-xylenol, dichlorophene, hexachlorophene, providon iodide, parabens, in particular alkyl parabens, such as methyl, ethyl, propyl or butyl paraben, benzyl paraben, acids, such as sorbic acid, benzoic acid and their salts, quaternary ammonia compounds, such as alkonium salts, e.g. bromides, benzalkonium salts, such as a chloride or a bromide, cetrimonium salts, such as a bromide, phenoalkecinium salts, such as a phenododecinium bromide, cetylpyridinium chloride and other salts, mercury compounds, such as phenyl mercury acetate, borate or nitrate, thiomersal, chlorohexidine or its gluconate or any antibiotically active compound of biological origin or any mixture of them.

This compound is preferably added in such an amount that it reduces the amount of bacteria in accordance with the following rule: reduction of the number of bacteria with the addition of 1,000,000 germs per gram of total mass of the composition to less than 100 for the case of aerobic bacteria or to less than 10 in the case of enterobacteria and to less than 1 in the case of Pseudomonas aeruginosa or Staphylococcus aureus after four days. Preferably this compound is however added in an amount so that this reduction is achieved after three days and most preferably after one day.

With regard to individual compounds the amounts stated in the following are generally suitable for obtaining such a reduction.

Short-chain alcohols, preferably ethyl alcohol, propyl alcohol, butyl alcohol or benzyl alcohol: up to 10% wt., more preferably up to 5% wt. and most preferably in the range between 0.5 and 3% wt., referred to the total composition, wherein for chlorobutanol a range from 0.3 to 0.6% wt. is especially preferred;
parabens, in particular methyl paraben: 0.05 to 0.2% wt. referred to the total composition and for propylparaben 0.002 to 0.02% wt. is particularly preferred;
sorbic acid: 0.05 to 0.2% wt., benzoic acid, 0.1 to 0.5% wt.;
phenols, trichlosan: 0.1 to 0.3% wt.;
chlorohexidine: 0.01 to 0.05% wt., referred in each case to the total composition.

A further additional constituent of the composition according to the invention can be an antioxidation agent. According to the invention any antioxidation agent can be used which is compatible with the main components of the composition according to the invention.

The at least one antioxidation agent is preferably used in an amount which reduces the oxidation index to less than 100% per six months, preferably the rise in the oxidation index is reduced to less than 100% per 12 months and especially preferably to less than 50% per 12 months. The antioxidation agent used according to the invention can be selected from the following group:

Synthetic phenolic antioxidation agents, such as butylated hydroxyanisol (BHA), butylated hydroxytoluol (BHT) and di-t-butylphenol (LY178002, LY256548, HWA-131, BF-389, CE-986, PD-127443, E-5119, BI-L-239XX), tertiary butylhydroquinone (TBHQ), propyl galeate (PG), 1-O-hexyl-2,3,5-trimethyl hydroquinone (HTHQ), aromatic amines (diphenylamine, p-alkylthio-o-anisidine, ethylene diamine derivatives, carbazol, tetrahydroindenoindol), phenols and phenolic acids (guaiacol, hydroquinone, vanillin, gallic acids and their esters, protocatechuic acid, quinic acid, syringic acid, ellagic acid, salicylic acid, nordihydroguaiaretic acid (NDGA) eugenol); tocopherols (including tocopherol (alpha, beta, gamma, delta) and their derivatives, such as tocopheryl acylate (e.g. acetate, laurate, myristate, palmitate, oleate, linoleate, etc. or any other suitable tocopheryl lipoate), tocopheryl POE succinate; Trolox and corresponding amide compounds and thiocarboxamide analogues; ascorbic acid and its salts, isoascorbate, (2 or 3 or 6)-o-alkyl ascorbic acids, ascorbic esters (e.g. 6-o-lauroyl, myristoyl, palmitoyl, oleyl or linoleoyl-L-ascorbic acid, etc.); non-steroidal inflammation inhibiting agents (NSAID) such as indomethacin, diclotenac, mefenamic acid, flutenamic acid, phenyl butazone, oxyphenbutazone, acetyl salicylic acid, naproxen, diflunisal, ibuprofen, ketoprofen, piroxicam, penicillamine, penicillamine disulphide, primaquin, quinacrine, chloroquine, hydroxychloroquine, azathioprine, phenobarbital, acetaminphen), aminosalicylic acids and derivatives; methotrexate, probucol, antiarrhythmic agents (amiodarone, apridin, asocainol), am broxol, tamoxifen, b-hydroxytamoxifen; calcium antagonists (nifedipine, nisoldipine, nimodipine, nicardipine, nilvadipine); beta receptor blockers (atenolol, propanolol, nebivolol), sodium bisulphite, sodium metabisulphite, thiourea; chelating agents, such as EDTA, GDTA, desferral; various endogenic defence systems, such as transferrin, lactoferrin, ferritin, caeruloplasmin, haptoglobin, haemopexin, albumin, glucose, ubichinol-10); enzymatic antioxidation agents such as superoxidismutase and metal complexes with a similar activity, including catalase, glutathione peroxidase, and less complex molecules such as beta-carotene, bilirubin, uric acid, flavonoids (flavones, flavonols, flavonones, flavanonals, chacones, anthocyanins), N-acetylcystein, mesna, glutathione, thiohistidine derivatives, triazoles; tannins, cinnamic acid, hydroxy cinnamic acid and its esters (coumaric acid and esters, caffeine acid and esters, ferulic acid (iso-)-chlorogenic acid, sinapic acid); spice extracts (e.g. from cloves, cinnamon, sage, rosemary, nutmeg blossom, oregano, clove pepper, nutmeg); carnosic acid, carnosol, carsolic acid; rosemarinic acid, rosemary diphenol, gentisinic acid, ferulic acid, oatmeal extracts, such as avenanthramide 1 and 2, thioether, dithioether, sulphoxides, tetraalcyl thiuram disulphide; phytic acid, steroid derivatives (e.g. U74006F); tryptophane metabolites (e.g. 3-hydroxykynurenine, 3-hydroxy anthranilic acid); and organochalcogenides.

Although the amount of antioxidation agent depends on the respective formulations of the composition according to the invention, general ranges can be stated for some of the preferred antioxidation agents, in which the desired germ reduction can be obtained. For BHA or BHT this concentration is from 0.001 to 2% wt., more preferably between 0.025 and 0.2% wt. and most preferably between 0.005 and 0.02% wt., referred in each case to the total composition, for TBHQ and PG between 0.01 and 2% wt., more preferably between 0.005 and 0.2% wt. and most preferably between 0.01 and 0.02% wt., for the tocopherols between 0.005 and 5% wt., more preferably between 0.01 and 0.5% wt. and most preferably between 0.05 and 0.075% wt., for ascorbic acid ester between 0.001 and 5% wt., more preferably between 0.005 and 0.5% wt. and most preferably between 0.01 and 0.15% wt., for ascorbic acid between 0.001 and 5% wt., more preferably between 0.005 and 0.5% wt. and most preferably between 0.01 and 0.1% wt., for sodium bisulphite or sodium metabisulphite between 0.001 and 5% wt., more preferably between 0.005 and 0.5% wt. and most preferably between 0.01 and 0.15% wt., for thiourea between 0.0001 and 2% wt., more preferably between 0.0005 and 0.2% wt. and most preferably between 0.001 and 0.01% wt., typically 0.005% wt., for cystein between 0.01 and 5% wt., more preferably between 0.05 and 2% wt. and most preferably between 0.1 and 1.0% wt., typically 0.5% wt., for monothioglycerol between 0.01 and 5% wt., more preferably between 0.05 and 2% wt. and most preferably between 0.1 and 1.0% wt., typically 0.5% wt., for NDGA between 0.0005 and 2% wt., more preferably between 0.001 and 0.2% wt. and most preferably between 0.005 and 0.002% wt., typically 0.01% wt., for glutathione between 0.005 and 5% wt., more preferably between 0.01 and 0.5% wt. and most preferably between 0.05 and 0.2% wt., typically 0.1% wt., for EDTA between 0.001 and 5% wt., more preferably between 0.005 and 0.5% wt. and most preferably between 0.01 and 0.2% wt., typically between 0.05 and 0.075% wt., for citric acid between 0.001 and 5% wt., more preferably between 0.005 and 3% wt. and most preferably between 0.01 and 0.2% wt., typically between 0.3 and 2% wt., referred in each case to the total composition.

A further optional constituent of the composition according to the invention is a consistency generator. A consistency generator is a compound which can influence the expansion rate of the composition according to the invention. In the composition according to the invention consistency generators are preferred which increase the expansion rate from the dry state to the state of full solvation by at least a factor of ten.

Such consistency generators are known to the art and in particular comprise pharmaceutically acceptable hydrophilic polymers, such as partially etherified cellulose derivatives, comprising carboxymethyl cellulose, hydroxyethyl cellulose, hydroxypropyl cellulose, hydroxypropylmethyl cellulose, hydroxymethyl cellulose or methyl cellulose, completely synthetic hydrophilic polymers, comprising polyacrylates, polymethacrylates, polyhydroxyethylmethacrylates, polyhydroxypropylmethacrylates, polyhydroxypropylmethylacrylates, polyacrylonitrile, methallylsulphonate, polyethylene, polyoxyethylene, polyethyleneglycols, polyethyleneglycol lactide, polyethyleneglycol diacrylate, polyvinylpyrrolidone, polyvinyl alcohols, polypropylmethacrylamide, polypropylene fumarate-co-ethylene glycol, polyoxamers, polyaspartamide, hydrazine cross-linked hyalurone acid, silicone, natural rubber, comprising alginates, carageenan, guar gum, gelatine, tragacanth, pectin, xanthan, chitosan, collagen, agarose, mixtures of them and derivatives or copolymers of them.

These consistency generators can be used on their own or in a mixture and are preferably used in an amount from 0.05 to 10% wt., referred to the total composition, more preferably from 0.1 to 5% wt., more preferably from 0.25 to 3.5% wt. and most preferably in the range from 0.5 to 2% wt.

Apart from the further optional constituents mentioned above, the composition according to the invention can contain further additives, such as cryogenic protective substances, flow promoters, expansion promoters, stabilisers, dyes, etc., provided these substances do not impair the composition according to the invention.

The composition according to the invention described above is suitable for the formation of liposomes. These can, for example, be formed in that the composition according to the invention is suspended in a physiologically acceptable buffer, such as a phosphate buffer, a citrate buffer, an acetate buffer or similar. The molarity of the buffer lies in the range from 10 mM to 1,000 mM, the pH value is preferably in the range between 2 and 9 and more preferably in the range between 3 and 6. The process steps required for forming liposomes are familiar to the person skilled in the art. By varying the method of producing liposomes when using the liposome-forming composition of this invention, liposomes of a variable size can be obtained, such as from 60 to 1,200 nm, preferably from 100 to 1,000 nm.

The formulations which contain the liposomes described above can be present in various ways depending on the intended method of administration. Examples of such formulations are, for example, lyophilised compositions obtained by the lyophilisation of the above compositions, suspended in a buffer, wherein these lyophilisates can be further processed in various ways. Examples of such further processing are the introduction into a hard gelatine capsule, formation into a tablet, in particular to a tablet with an acid-resistant coating.

The liposomes produced in this way are particularly suitable for the transport of active substances based on peptides, wherein these active substances are preferably peptides with a molecular weight in the range from 200 to 100,000 Da, such as parathyroid hormone, salmon calcitonin, GCSF, octreotide, hGH, insulin and similar substances. Active substances particularly preferred, which can be formulated with the compositions according to the invention to form liposomes containing the active substance, are octreotide, calcitonin, parathyroid hormone and somatropin.

The active substances to be introduced into the liposomes may either be originally already present in the composition according to the invention or may be added during the formation of the liposomes. The amount of active substance, which is used in the respective formulations, can of course be varied in relation to the desired purpose. However, it has been established that particularly stable and efficient liposomes containing the active substance can be obtained if the ratio between the total molarity of lipid and tetraether lipid to the mass of the active substance lies in the range from 0.01 to 0.2 micromol/microgram, more preferably in the range from 0.03 micromol/microgram to 0.15 micromol/microgram and most preferably between 0.05 micromol/microgram and 0.1 micromol/microgram.

The preferred values stated above for the ratio of total lipid to active substance demonstrate a further advantage of this invention. In particular when taken orally, the compositions of the invention facilitate an oral availability of the active substances, which can also be controlled via the ratio of total lipid to active substance (preferably a peptide active substance). The values given above show in view of the formulations formed from conventional lipids that with the use of the mixture according to the invention of a double-layer forming lipid and a double-layer spanning tetraether lipid, less total lipid content is necessary to achieve a good oral availability. In this respect the following tests demonstrate that with the use of DPPC less total lipid is required than when DSPC is used for the same ratio of double-layer forming lipid to double-layer spanning tetraether lipid, wherein however, also with the use of DSPC, a more significant increase in the oral availability is obtained than with conventional compositions.

This invention is further explained by the following examples:

The determination of the bioavailablility of liposomally administered octreotides per os in rats occurred in the conventional way, for example as described below.

Wistar rats (with approximately 250 g body weight) fast for 12 hours and receive 0.5 ml of the aqueous mixture of the composition according to the invention by pharyngeal probe so that the administered amount of octreotide is 100 μg per animal. As a comparative test 100 μg of free octreotide was dissolved in 0.5 ml of PBS and administered via pharyngeal probe. Blood samples were taken after 30, 60, 120 and 240 min. retroorbitally. An i.v. injection of 2 μg of octreotide per animal provided a control in order to determine the absolute bioavailability. An absolute bioavailability of 0.5% was measured for free octreotide. Six animals were tested for each formulation.

### EXAMPLE 1

Oral administration of octreotide with liposomes with the composition of DSPC and GCTE, with a molar ratio DSPC:GCTE of 3:1 and a ratio of the molarity of the total lipid to the mass of the peptide of 0.097 μmol/μg as well as 0.038 μmol/μg and oral administration of octreotide with liposomes with the composition of DPPC and GCTE, with a molar ratio of DPPC:GCTE of 3:1 and a ratio of the molarity of the total lipid to the mass of the peptide of 0.013 μmol/μg and 0.029 μmol/μg. The respective gain factors of the absolute bioavailability and the absolute bioavailability are shown in the following table:

| Octreotide oral | | Abs. | |
|---|---|---|---|
| Dose 0.1 | Lipid/peptide | bioavailability | Gain |
| mg/animal | (μmol/μg) | (%) | factor |
| Free | 0 | 0.5 | 1 |
| DSPC/GCTE 3/1 | 0.038 | 1.8 | 3.6 |
| DPPC/GCTE 3/1 | 0.097 | 6.8 | 13.7 |
| DSPC/GCTE 3/1 | 0.013 | 5.6 | 11.2 |
| DSPC/GCTE 3/1 | 0.029 | 12.9 | 25.9 |

These results are summarised in the following chart.

[image]

### EXAMPLE 2

Oral administration of octreotide with liposomes with the composition of DSPC and GCTE and AFI, with a molar ratio of DSPC:GCTE:AF1 of 6:1.5:0.5 and a ratio of the molarity of the total lipid to the mass of the peptide of 0.081 μmol/μg. The gain factor of the absolute bioavailability of octreotide in this mixture compared to the absolute bioavailability of free octreotide is 12 and the absolute bioavailability is 6%. These results are summarised in the following chart.

[image]

### EXAMPLE 3

Oral administration of octreotide with liposomes with the composition of DPPC and GCTE, with a molar ratio of DPPC:GCTE of 3:1 and a ratio of the molarity of the total lipia to the mass of the peptide of 0.013 μmol/μg, with a molar ratio of DPPC:GCTE of 2:1 and a ratio of the molarity of the total lipid to the mass of the peptide of 0.007 μmol/μg, and with a molar ratio of DPPC:GCTE of 4:1 and a ratio of the molarity of the total lipid lo the mass of the peptide of 0.011 μmol/μg. The gain factor of the absolute bioavailability of octreotide in this mixture compared to the absolute bioavailability of free octreotide is 3.6, 3.2 and 1.6 and the absolute bioavailability Is 7.2%, 6.4% and 3.2%. These results are summarised in the following charts.

[image]

It has been shown that a preferred molar mixing ratio of lipid to tetraether lipid lies in the lower range, preferably from 3:1 to 2:1, especially preferably 2.5:1. Preferably the lipid here is cholesterol and the tetraether lipid is a tetraether lipid with the phosphatidyl choline header group.

It is preferable to add a penetration promoter to the liposome-forming composition.

It is preferable to use a cholate derivative as the penetration promoter, chenodeoxycholic acid and ursodeoxycholic acid are very preferable or also mixtures of the two.

It is preferable to add the penetration promoter so that it is enclosed together with the active substance by the liposome and/or it is also preferable that the penetration promoter is incorporated in the liposome membrane which in turn encloses the active substance.

For peptides with a therapeutic daily dose of 10-100 μg/kg, it is preferable to select the mass ratio of penetration promoter to peptide larger than 12.5, but less than 1000.

For peptides with a therapeutic daily dose of 10-100 μg/kg, it is preferable to select the mass ratio of peptide to liposomal envelope larger than 0.1, but less than 10.

1. Liposome-forming composition, containing at least one lipid, which is capable of forming a double layer, and tetraether lipid which spans the double layer.

2. Composition according to claim 1, further containing at least one antioxidation agent and/or at least one preservative and/or one consistency generator.

3. Composition according to claim 1, further containing a pharmaceutical active substance.

4. Composition according to claim 3, wherein the pharmaceutical active substance is a peptide active substance.

5. Composition according to claim 1, wherein the molar ratio of lipid to tetraether lipid lies in the range from 2.5:1 to 5:1.

6. Composition according to claim 1, wherein the lipid is DSPC or DPPC and the tetraether lipid is GCTE.

7. Composition according to claim 1, wherein the lipid is DSPC and the tetraether lipid is a mixture of GCTE and AF1.

8. canceled

9. Liposomes, obtainable by suspending a composition according to claim 1 in an aqueous medium.

10. Liposomes according to claim 9, wherein the liposomes contain a peptide active substance.

11. Liposomes according to claim 10, wherein the ratio between the total molarity of lipid and tetralipid to the mass of the peptide active substance lies in the range from 0.005 to 0.2 micromol/microgram.
